(19) United States
(12) Patent Application Publication
(10) Pub. No.: US 2019/0021713 A1
(43) Pub. Date: Jan. 24, 2019

(54) LAPAROSCOPIC TOOL COVER AND METHOD OF USE

(71) Applicant: Douglas W. Walker, Fort Worth, TX (US)

(72) Inventor: Douglas W. Walker, Fort Worth, TX (US)

(21) Appl. No.: 16/041,368

(22) Filed: Jul. 20, 2018

Related U.S. Application Data
(60) Provisional application No. 62/534,828, filed on Jul. 20, 2017.

(51) Int. Cl. A61B 17/00; A61B 17/29; A61B 46/10

(52) U.S. Cl. CPC A61B 17/00234; A61B 2017/2829; A61B 46/10; A61B 17/29

(57) ABSTRACT

A laparoscopic tool sheath for covering a body of a laparoscope, the cover includes an elongated body, having a rubber base; and a highly absorbable top coating; a central passageway extending from a distal end of the elongated body to a proximal end of the elongated body; a distal lens integral to the distal end of the elongated body; the cover is able to quickly and easily be removed from the laparoscopic tool for disposal.

### BACKGROUND

### 1. Field of the Invention

The present invention relates generally to surgical tools and equipment, and more specifically, to a laparoscopic tool cover for easy installation and removal of the cover. The cover further provides improved visualization through an anti-fog/smudge lens.

### 2. Description of Related Art

Laparoscopic tool systems are well known in the art and are commonly used during minimally invasive surgeries, providing a modern surgical technique in which operations are performed through small incisions into the body. For example, FIG. 1 depicts a conventional laparoscopic tool system 101 having a light source 105, a camera 107, and a scope 109. During use, scope 109 is inserted into the body, wherein camera 107 provides a visual representation to the user.

One of the problems commonly associated with system 101 is maintenance of a clear visual field. For example, common practice requires continuous removal of the laparoscope and manually cleaning of the distal end due to rundown of blood/body fluids down the body of the scope.

Accordingly, although great strides have been made in the area of laparoscopic tool systems, many shortcomings remain.

### DESCRIPTION OF THE DRAWINGS

The novel features believed characteristic of the embodiments of the present application are set forth in the appended claims. However, the embodiments themselves, as well as a preferred mode of use, and further objectives and advantages thereof, will best be understood by reference to the following detailed description when read in conjunction with the accompanying drawings, wherein:

FIG. 1 is a simplified side view of a common laparoscopic tool system;

FIG. 2 is a simplified side view of a laparoscopic tool system with a cover in accordance with a preferred embodiment of the present application;

FIGS. 3A-3C are simplified side views of an installment of the cover of FIG. 2;

FIG. 4 is a cross sectional view of the cover of FIG. 2 secured to a scope;

FIGS. 5A and 5B depict alternative embodiments of the cover of FIG. 2;

FIG. 6 is a flowchart of the method associated with FIG. 2;

FIG. 7 is a side view of a laparoscopic tool system in accordance with an alternative embodiment of the present application; and

FIG. 8 is a cross sectional view of the cover and irrigation tubing of FIG. 7.

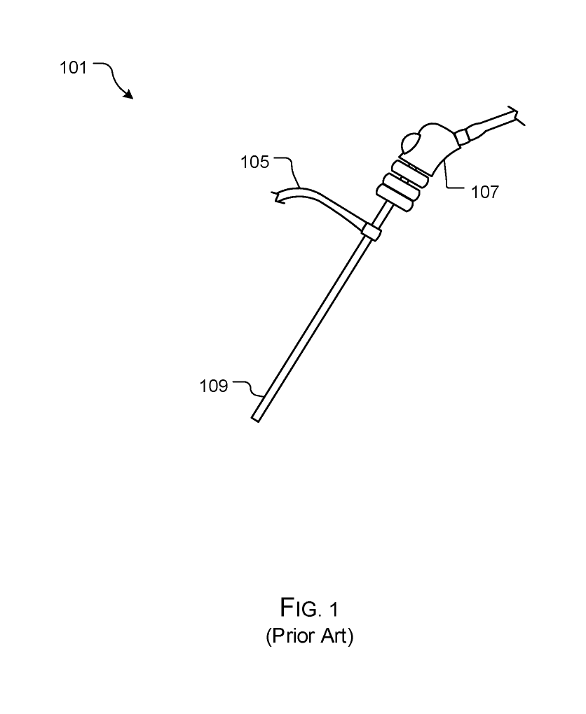

While the system and method of use of the present application is susceptible to various modifications and alternative forms, specific embodiments thereof have been shown by way of example in the drawings and are herein described in detail. It should be understood, however, that the description herein of specific embodiments is not intended to limit the invention to the particular embodiment disclosed, but on the contrary, the intention is to cover all modifications, equivalents, and alternatives falling within the spirit and scope of the present application as defined by the appended claims.

### DETAILED DESCRIPTION OF THE PREFERRED EMBODIMENT

Illustrative embodiments of the system and method of use of the present application are provided below. It will of course be appreciated that in the development of any actual embodiment, numerous implementation-specific decisions will be made to achieve the developer's specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

The system and method of use in accordance with the present application overcomes one or more of the above-discussed problems commonly associated with conventional laparoscopic tool systems. Specifically, the present invention provides a cover that is convenient to attach to and remove from a scope of a laparoscopic tool. These and other unique features of the system and method of use are discussed below and illustrated in the accompanying drawings.

The system and method of use will be understood, both as to its structure and operation, from the accompanying drawings, taken in conjunction with the accompanying description. Several embodiments of the system are presented herein. It should be understood that various components, parts, and features of the different embodiments may be combined together and/or interchanged with one another, all of which are within the scope of the present application, even though not all variations and particular embodiments are shown in the drawings. It should also be understood that the mixing and matching of features, elements, and/or functions between various embodiments is expressly contemplated herein so that one of ordinary skill in the art would appreciate from this disclosure that the features, elements, and/or functions of one embodiment may be incorporated into another embodiment as appropriate, unless described otherwise.

The preferred embodiment herein described is not intended to be exhaustive or to limit the invention to the precise form disclosed. It is chosen and described to explain the principles of the invention and its application and practical use to enable others skilled in the art to follow its teachings.

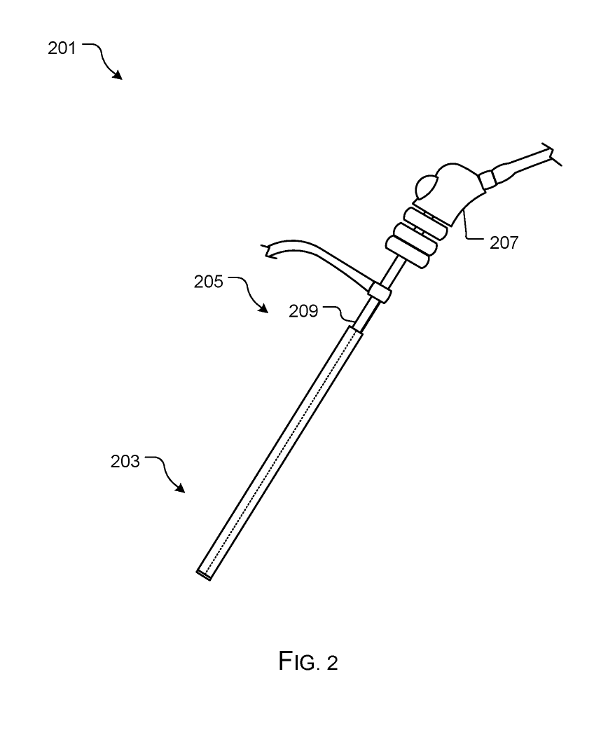

Referring now to the drawings wherein like reference characters identify corresponding or similar elements throughout the several views, FIG. 2 depicts a simplified side view of a laparoscope tool system 201 having a cover 203 in accordance with a preferred embodiment of the present application. It will be appreciated that system 201 overcomes one or more of the above-listed problems commonly associated with conventional laparoscope tool systems.

In the contemplated embodiment, system 201 includes a tool 205 having at a camera 207 and a scope 209 configured to receive cover 203. During use, system 201 is used during minimally invasive surgical procedures.

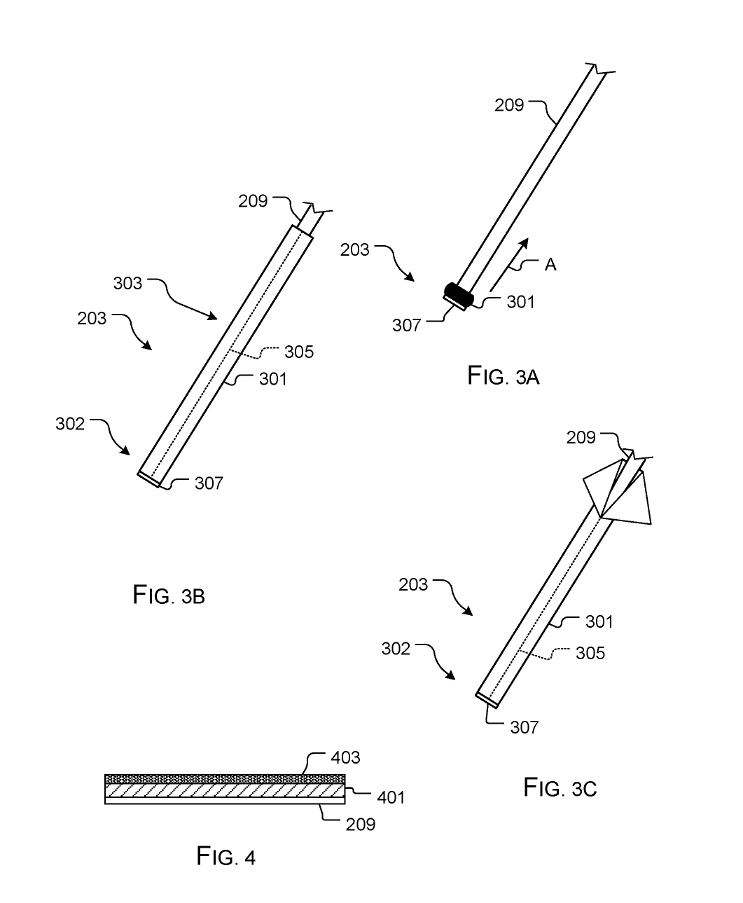

As shown in FIGS. 3A-3C, cover 203 includes an elongated body 301 configured to be secured to scope 209 be being rolled into place, as shown in FIG. 3A with arrow A. Body 301 encompasses a central passageway extending from a distal end 302 of the elongated body to a proximal end 303 of the elongated body. It should be appreciated that this configuration allows for cover 203 to be quickly and snugly secured to scope 209. It should be appreciated that cover 203 can be sized appropriately to fit a variety of scope sizes, including but not limited to a 30/45 cm length scope. Body 301 further includes a perforated seam 305 providing for quick and convenient removal of cover 203 for disposal, as shown in FIG. 3C.

In the preferred embodiment, cover 203 further includes a distal lens 307 integral with body 301. Lens 307 is configured to rest over the distal end of scope 209 and provide a clear view into a body cavity. It should be appreciated that lens 307 can be composed of a glare resistant, water resistant, fog resistant, and/or smudge resistant material.

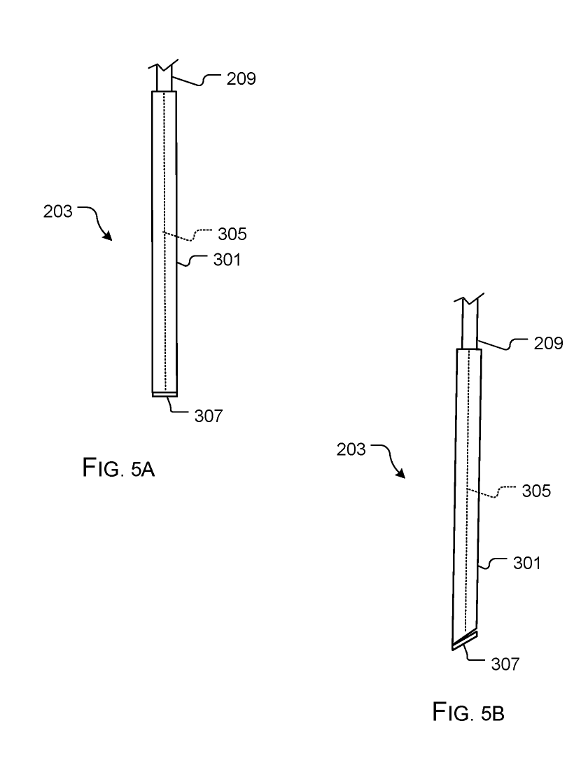

In FIG. 4, a cross sectional side view depicts body 301 secured to scope 209. In the preferred embodiment, body 301 is composed of at least two layers, wherein the first layer 401 is composed of a rubber and the second layer 403 is composed of a top coating. It should be appreciated that the top coating consists of a highly absorbable material, thereby maintaining a clear visual field by absorbing blood/body fluids that typically obscure the scope lens.

In FIGS. 5A and 5B, side views demonstrate the various embodiments of cover 203. In one embodiment, as shown in FIG. 5A, lens 307 is positioned at a 0 degree angle, as is a common scope configuration. In an alternative embodiment, as shown in FIG. 5B, lens 307 is positioned at a 30 degree angle. It should be appreciated that various embodiments contemplate other angles as well.

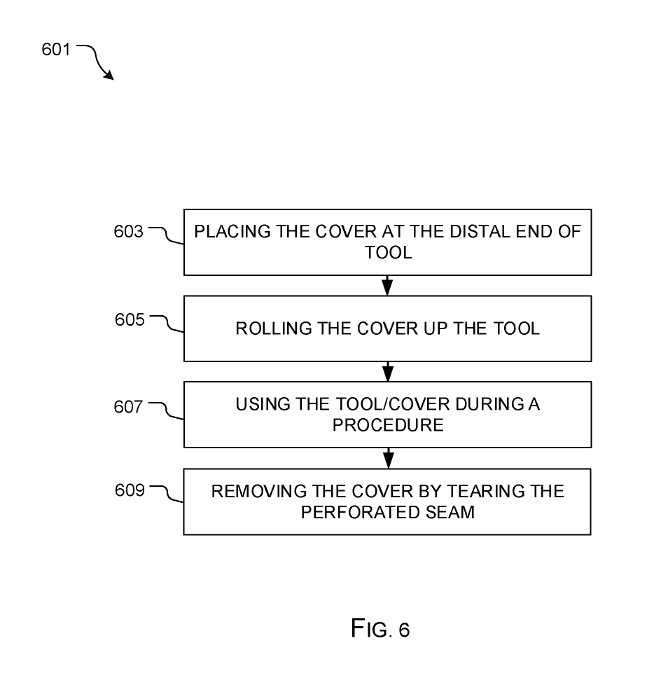

In FIG. 6, a flowchart 601 depicts a method of FIG. 2. The cover is secured to the distal end of the scope and rolled up the scope to be secured into a useable position, as shown with boxes 603, 605. The laparoscope tool and cover are then used as needed during a procedure, as shown with box 607. After the procedure is completed, the cover is removed for disposal by tearing the perforated seam, as shown with box 609.

It should be appreciated that one of the unique features believed characteristic of the present application is the ability to maintain clear visual fields throughout procedures, as well as the configuration of the body and lens cover, allowing for the cover to be easily attached and removed from the body of the laparoscope.

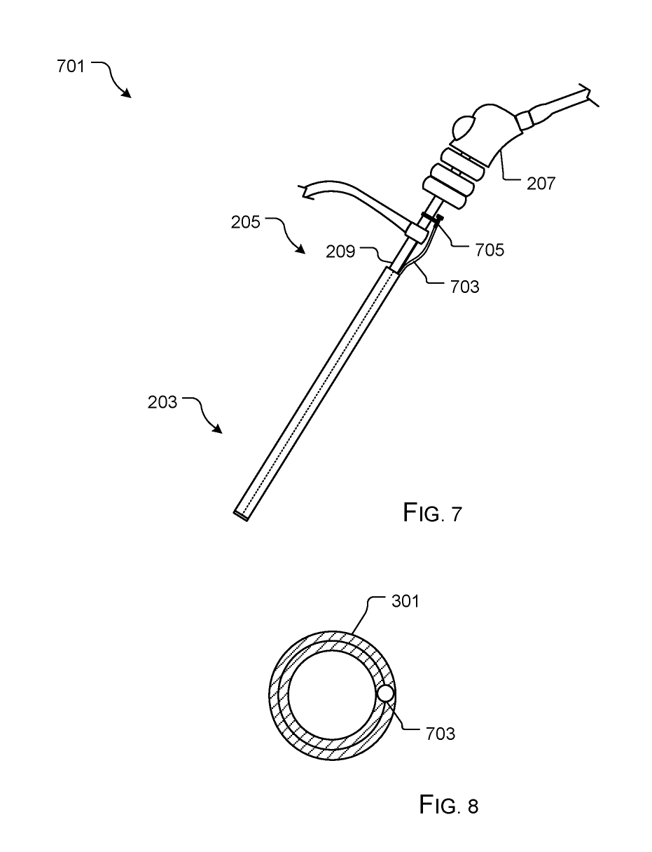

In FIGS. 7 and 8, an alternative embodiment of a laparoscope tool system 701 is shown. It should be appreciated that the features discussed above could be incorporated into system 701 as desired. System 701 includes an irrigation tubing 703 composed of a malleable plastic, and attached distally to a syringe (not shown) and attaching to the main scope body via a snap connector 705. As shown in FIG. 8, the irrigation tubing 703 is embedded within the absorbable body 301.

The particular embodiments disclosed above are illustrative only, as the embodiments may be modified and practiced in different but equivalent manners apparent to those skilled in the art having the benefit of the teachings herein. It is therefore evident that the particular embodiments disclosed above may be altered or modified, and all such variations are considered within the scope and spirit of the application. Accordingly, the protection sought herein is as set forth in the description. Although the present embodiments are shown above, they are not limited to just these embodiments, but are amenable to various changes and modifications without departing from the spirit thereof.

What is claimed is:

1. A laparoscopic tool cover for covering a shaft of a tool, the cover comprising:
an elongated body, having:
a rubber base; and
a highly absorbable top coating;
a central passageway extending from a distal end of the elongated body to a proximal end of the elongated body;
a distal lens integral to the distal end of the elongated body;
wherein the cover is configured to be removed from the laparoscopic tool for disposal.

2. The cover of claim 1, wherein the distal lens is comprised of an anti fog/water resistant/smudge resistant/glare resistant material.

3. The cover of claim 1, wherein the distal lens is positioned at a 0 degree angle associated with the laparoscopic tool.

4. The cover of claim 1, wherein the distal lens is positioned at a 30 degree angle associated with the laparoscopic tool.

5. The cover of claim 1, wherein the elongated body further comprises:
a perforated seam extending from the distal end to the proximal end;
wherein the perforated seam allows for easy removal of the cover for disposal.

6. The cover of claim 1, wherein the top coating is composed of a highly absorbent material.

7. The cover of claim 1, wherein the cover is configured to fit a variety of sizes including, but not limited to 30/45 cm length scopes.

8. The cover of claim 1, further comprising: an irrigation tubing embedded within the elongated body.
